(19) United States
(12) Patent Application Publication
(10) Pub. No.: US 2019/0121939 A1
(43) Pub. Date: Apr. 25, 2019

(54) DAISY CHAIN FOR PHASING POLYPLOID

(71) Applicant: INTERNATIONAL BUSINESS MACHINES CORPORATION, Armonk, NY (US)

(72) Inventors: Laxmi Parida, Mohegan Lake, NY (US); Filippo Utro, Pleasantville, NY (US)

(21) Appl. No.: 15/793,578

(22) Filed: Oct. 25, 2017

(51) Int. Cl. G06F 19/22; G06F 19/18; C12Q 1/68

(52) U.S. Cl. CPC G06F 19/22; G06F 19/18; C12Q 2600/172; C12Q 1/6827; C12Q 2545/101; C12Q 1/6811

(57) ABSTRACT

A computer-implemented method is provided for phasing polyploids. The method includes assembling, by a processor, haplotypes in polyploid genomes of two or more sample organisms. The method further includes performing, by the processor, haplotype phasing on the haplotypes using a haplotype phasing process having multiple phases applied to ploid samples corresponding to the haplotypes to determine which parent chromosomes possess which of the haplotypes. The step of performing haplotype phasing includes (i) reducing, in each of the multiple phases, a ploidy p to a floor value (p/2), (ii) assigning variables in at least some of the multiple phases such that resulting intermediate ones of the ploid samples co-occur multiple times in the at least some of the multiple phases, and (iii) linking, in each of the multiple phases, the ploid samples together using one or more daisy chain models and daisy chain linkage criteria.

### BACKGROUND

### Technical Field

The present invention relates generally to polyploid phasing and, in particular, to a daisy chain for phasing a polyploid.

### Description of the Related Art

Organisms typically possess multiple copies of the same chromosome. Many species in nature are polyploid, which means the species has 2 or more copies of the same chromosomes. Examples of polyploid species include triploids (e.g., seedless watermelons), tetraploids (e.g., Salmonida fish), pentaploids (e.g., Kenai Birch), hexaploid (e.g., wheat, kiwifruit), octaploids or octoploids (e.g., Acipenser, dahlias), decaploids (e.g., certain strawberries), and dodecaploids (e.g., Celosia argentea, Spartina angilica, and Xenopus ruwenzoriensis). Polyploidy is common in plants and is also observed in some animals. Moreover, some human tissue can be polyploidy (e.g., human muscle tissues, human liver tissues, and human bone marrow).

However, genotyping methods cannot separate these different copies of the same chromosome. Hence, there is a need for an approach for phasing polyploids that is capable of separating these different copies of the same chromosome.

### SUMMARY

According to an aspect of the present invention, a computer-implemented method is provided for phasing polyploids. The method includes assembling, by a processor, haplotypes in polyploid genomes of two or more sample organisms. The method further includes performing, by the processor, haplotype phasing on the haplotypes using a haplotype phasing process having multiple phases applied to ploid samples corresponding to the haplotypes to determine which parent chromosomes possess which of the haplotypes. The step of performing haplotype phasing includes reducing, in each of the multiple phases, a ploidy p to a floor value (p/2). The step of performing haplotype phasing further includes assigning variables in at least some of the multiple phases such that resulting intermediate ones of the ploid samples co-occur multiple times in the at least some of the multiple phases. The step of performing haplotype phasing also includes linking, in each of the multiple phases, the ploid samples together using one or more daisy chain models and daisy chain linkage criteria.

According to another aspect of the present invention, a computer program product is provided for phasing polyploids. The computer program product includes a non-transitory computer readable storage medium having program instructions embodied therewith. The program instructions are executable by a computer to cause the computer to perform a method. The method includes assembling, by a processor, haplotypes in polyploid genomes of two or more sample organisms. The method further includes performing, by the processor, haplotype phasing on the haplotypes using a haplotype phasing process having multiple phases applied to ploid samples corresponding to the haplotypes to determine which parent chromosomes possess which of the haplotypes. The step of performing haplotype phasing includes reducing, in each of the multiple phases, a ploidy p to a floor value (p/2). The step of performing haplotype phasing further includes assigning variables in at least some of the multiple phases such that resulting intermediate ones of the ploid samples co-occur multiple times in the at least some of the multiple phases. The step of performing haplotype phasing also includes linking, in each of the multiple phases, the ploid samples together using one or more daisy chain models and daisy chain linkage criteria.

According to yet another aspect of the present invention, a computer processing system is provided for phasing polyploids. The computer processing system includes a processor. The processor is configured to assemble haplotypes in polyploid genomes of two or more sample organisms. The processor is further configured to perform haplotype phasing on the haplotypes using a haplotype phasing process having multiple phases applied to ploid samples corresponding to the haplotypes to determine which parent chromosomes possess which of the haplotypes. The processor performs the haplotype phasing by reducing, in each of the multiple phases, a ploidy p to a floor value (p/2). The processor further performs the haplotype phasing by assigning variables in at least some of the multiple phases such that resulting intermediate ones of the ploid samples co-occur multiple times in the at least some of the multiple phases. The processor also performs the haplotype phasing by linking, in each of the multiple phases, the ploid samples together using one or more daisy chain models and daisy chain linkage criteria.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 shows an exemplary processing system to which the present invention may be applied, in accordance with an embodiment of the present invention;

FIG. 2 shows an exemplary system for phasing polyploids using a daisy chain, in accordance with an embodiment of the present invention;

FIG. 3 depicts an input matrix with three hexaploid organisms, in accordance with an embodiment of the present invention;

FIGS. 4-5 show an exemplary method for phasing a polyploid using a daisy chain, in accordance with an embodiment of the present invention; and

FIG. 6 shows exemplary daisy chains, in accordance with an embodiment of the present invention.

### DETAILED DESCRIPTION

The present invention is directed to a daisy chain for phasing a polyploid.

In accordance with one or more embodiments of the present invention, methods, systems and computer program products are provided for a daisy chain for phasing polyploids. A polyploid is an organism that has two or more copies of the same chromosome. For example, a kiwi is a polyploid that has five copies of a chromosome. In agriculture, certain traits of plants are desired over other traits. For example, a kiwi having a larger size and/or is evenly shaped is desirable over a kiwi that is smaller and/or has an oddly shape. Another example is a seedless fruit such as a seedless watermelon. When certain traits are deemed desirable, determining a particular part (or full) chromosome(s) that the trait is descendent from is required for repeating the trait in future progeny.

In an embodiment, the present invention can separate multiple copies of the chromosome. In an embodiment, the present invention can achieve such separation, where the ancestral haplotypes are more or less uniformly present (without peaks). In an embodiment, the present invention can achieve such separation for cases where the number of copies of the chromosome (i.e., its ploidy) is 2 or larger.

The determination of parent chromosomes possessing desired traits is sometimes referred to as “haplotype estimation” or “haplotype phasing”. A haplotype is a group of genes within an organism that was inherited together from a single parent. The word “haplotype” is derived from the word “haploid,” which describes cells with only one set of chromosomes, and from the word “genotype,” which refers to the genetic makeup of an organism. Haplotype phasing, typically, utilizes a process of statistical estimation of a haplotype from genotype data. In one or more embodiments of the present invention, a phasing of polyploids that uses a daisy chain to determine haplotypes is performed utilizing a non-naïve algorithm as described herein.

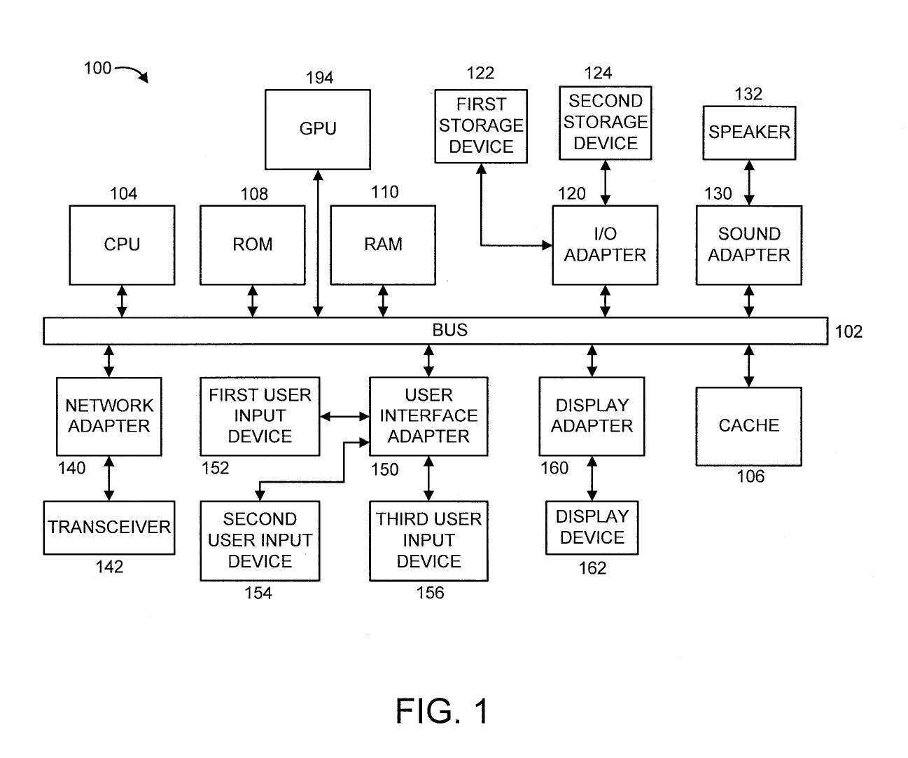

FIG. 1 shows an exemplary processing system 100 to which the invention principles may be applied, in accordance with an embodiment of the present invention. The processing system 100 includes at least one processor (CPU) 104 operatively coupled to other components via a system bus 102. A cache 106, a Read Only Memory (ROM) 108, a Random Access Memory (RAM) 110, an input/output (I/O) adapter 120, a sound adapter 130, a network adapter 140, a user interface adapter 150, and a display adapter 160, are operatively coupled to the system bus 102. At least one Graphics Processing Unit (GPU) 194 is operatively coupled to the system bus 102.

A first storage device 122 and a second storage device 124 are operatively coupled to system bus 102 by the I/O adapter 120. The storage devices 122 and 124 can be any of a disk storage device (e.g., a magnetic or optical disk storage device), a solid state magnetic device, and so forth. The storage devices 122 and 124 can be the same type of storage device or different types of storage devices.

A speaker 132 is operatively coupled to system bus 102 by the sound adapter 130. A transceiver 142 is operatively coupled to system bus 102 by network adapter 140. A display device 162 is operatively coupled to system bus 102 by display adapter 160.

A first user input device 152, a second user input device 154, and a third user input device 156 are operatively coupled to system bus 102 by user interface adapter 150. The user input devices 152, 154, and 156 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 152, 154, and 156 can be the same type of user input device or different types of user input devices. The user input devices 152, 154, and 156 are used to input and output information to and from system 100.

Of course, the processing system 100 may also include other elements (not shown), as readily contemplated by one of skill in the art, as well as omit certain elements. For example, various other input devices and/or output devices can be included in processing system 100, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 100 are readily contemplated by one of ordinary skill in the art given the teachings of the present invention provided herein.

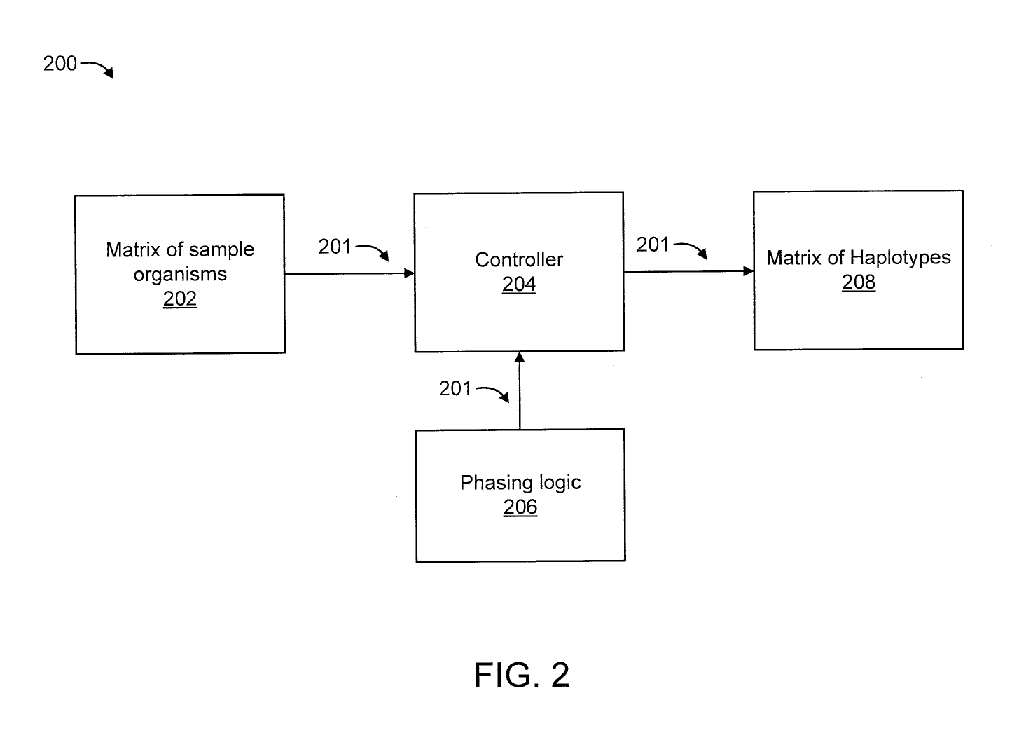

Moreover, it is to be appreciated that system 200 described below with respect to FIG. 2 is a system for implementing respective embodiments of the present invention. Part or all of processing system 100 may be implemented in one or more of the elements of system 200.

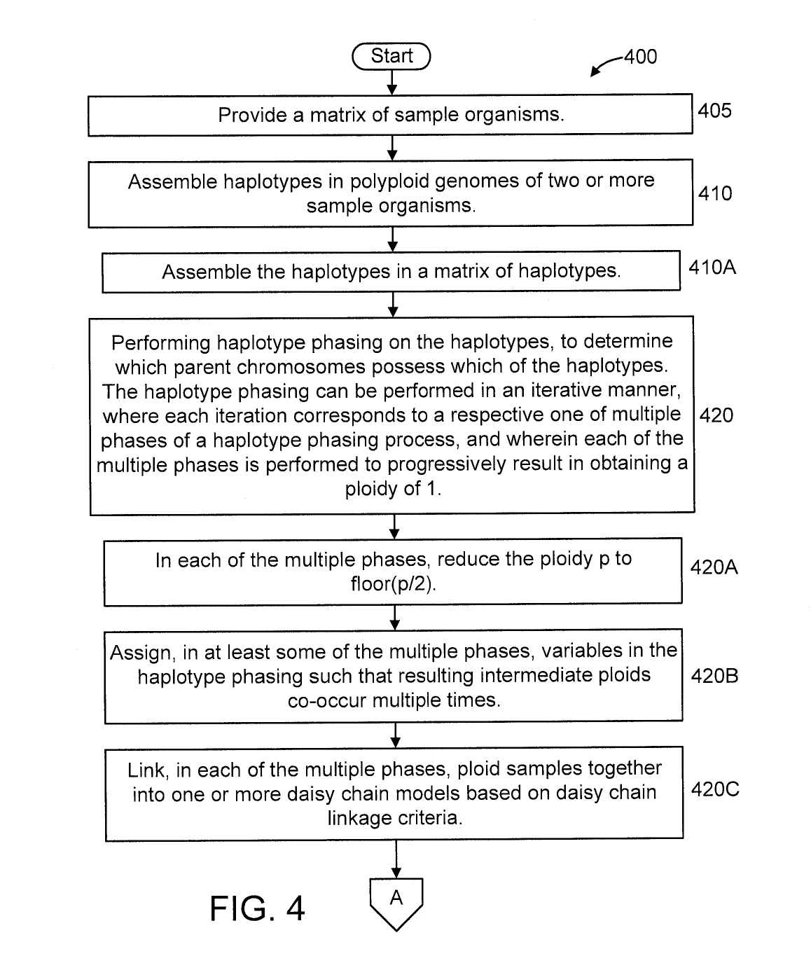

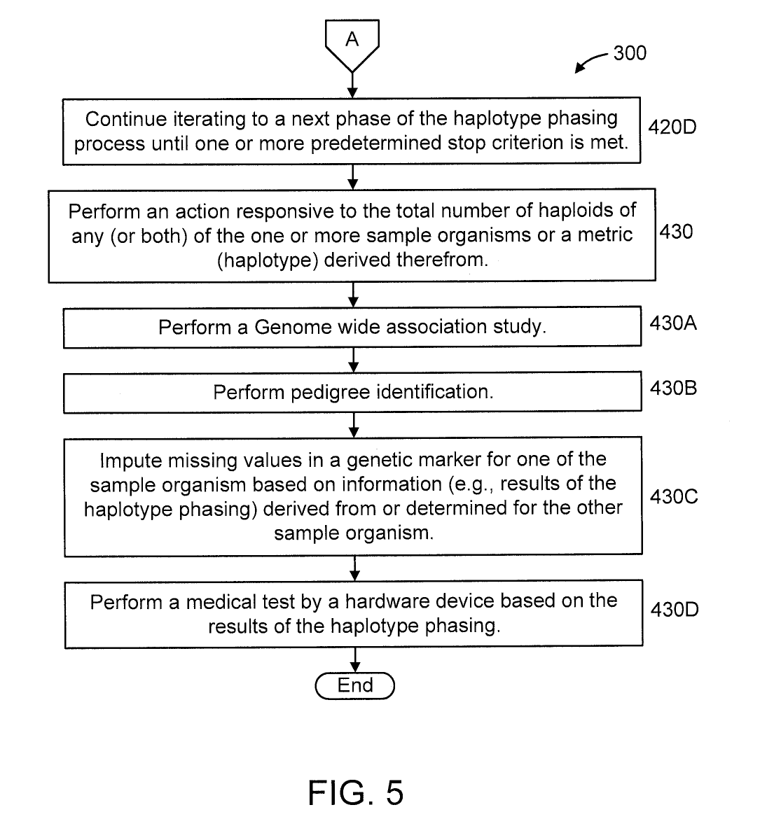

Further, it is to be appreciated that processing system 100 may perform at least part of the method described herein including, for example, at least part of method 400 of FIG. 4-5. Similarly, part or all of system 200 may be used to perform at least part of method 400 of FIGS. 4-5.

FIG. 2 shows an exemplary system 200 for phasing polyploids using a daisy chain, in accordance with an embodiment of the present invention. While the system 200 involves one or more matrices in order to perform polyploid phasing in accordance with an embodiment of the present invention, other data constructs can also be used in accordance with the teachings of the present invention while maintaining the spirit of the present invention, as readily appreciated by one of ordinary skill in the art, given the teachings of the present invention provided herein. That is, while system 200 is described using one or more matrices for the sake of illustration, other data constructs can also be used, while maintaining the spirit of the present invention.

The system 200 includes a matrix of sample organisms 202, a controller 204, phasing logic 206, and a matrix of haplotypes 208, configured and arranged as shown. The system 200 addresses the problem of phasing samples given a matrix of sample organisms 202, with such phasing employing a daisy chain.

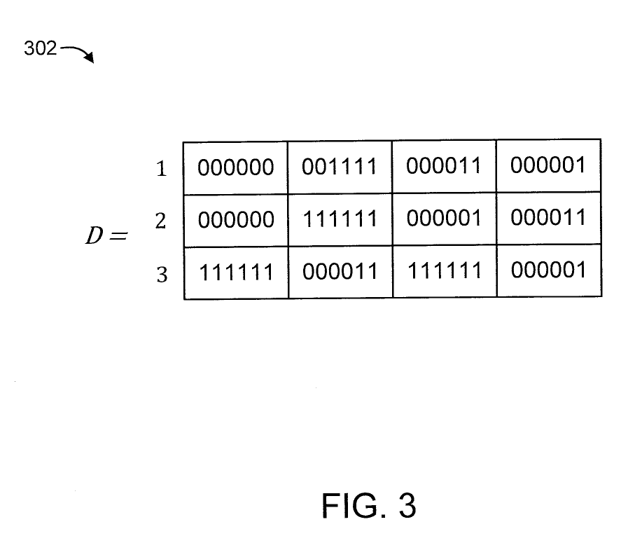

Within the matrix 202, there are rows that denote a sample organism and there are columns that represent an ordered sequence of single-nucleotide polymorphisms (SNPs). In one or more embodiments of the invention, the SNPs are assumed to be bi-allelic. FIG. 3 depicts an input matrix D 302 with three hexaploid organisms (a, b, c), in accordance with an embodiment of the present invention. In each column of the input matrix D, there are single-nucleotide polymorphisms (SNPs) which are represented by either a 0 or a 1. The SNPs show a 0 for a minor allele (i.e., minor gene variant) and a 1 for a major allele (i.e., major gene variant). In some embodiments, the SNPs can be represented as a 1 for minor alleles and a 0 for major alleles. The number of Os and Is in each column is based on the ploidy of the sample organisms. The organisms in the input matrix 302 are hexaploidy organisms with a total of six Os and Is such as 001111 and 000011. Of course, organisms having other ploidys can also be used, while maintaining the spirit of the present invention.

Hence, in an embodiment, each element of the input matrix 302 (i.e., each cell) is a genotype, defined as X. Coded genotype X and x₁, x₀, x₁, x_{L}, Xₚ, (X) of X are defined below. Each genotype is equivalent to a 3-tuple (triple):

$$X ≡ ( {x}_{1} , {x}_{0} , {x}_{l} ) , \\ where$$

$$\{ \begin{matrix}{x}_{1} & number of 1 ' s \\ {x}_{0} & number of 0 ' s \\ {x}_{l} & number of variables \\ {x}_{L} & is the set of variables and {x}_{l} = \vert {x}_{L} \vert \\ {X}_{p} & is the ploidy and {X}_{p} = {x}_{1} + {x}_{0} + {x}_{l} \\ \{ X \} & set coded by X\end{matrix}$$

With polyploid phasing, a matrix of sample organisms 202, such as the input matrix D 302, has the phasing logic applied iteratively until only monoploid rows remain or there is no change in D between one iteration and the next iteration of the algorithm.

The phasing logic 206 is utilized to implement the phasing of polyploids. In an embodiment, the phasing logic 206 performs algebraic phasing of polyploids. However, as noted above, other types of phasing can also be used. An input matrix D, such as the input matrix 302, where each row represents a sample and the ordered columns represent the ordered SNPs is utilized as a representation of a set of SNPs for multiple organisms (e.g., polyploids). Each entry of the matrix is a genotype of ploid k, i.e., a binary set of size k. The phasing process resolves the matrix D 302 in to the smallest number of haplotypes (or rows of ploidy 1).

An input matrix including two or more sample polyploid organisms are initialized by setting the ploidy of each row to be k, the input ploidy and for each ith row, <X>, the set Sₓ is initialized to the sample represented by that row with multiplicity k. A phasing process applied by phasing logic 206 (heuristics) is performed until all the rows have a ploidy of 1.

In the embodiment shown in FIG. 2, the elements thereof are interconnected by a bus(es)/network(s) 201. However, in other embodiments, other types of connections can also be used. Moreover, in an embodiment, at least one of the elements of system 200 is processor-based. Further, while one or more elements may be shown as separate elements, in other embodiments, these elements can be combined as one element. The converse is also applicable, where while one or more elements may be part of another element, in other embodiments, the one or more elements may be implemented as standalone elements. Moreover, one or more elements of FIG. 2 can be implemented in a cloud configuration including, for example, in a distributed configuration. Additionally, one or more elements in FIG. 2 may be implemented by a variety of devices, which include but are not limited to, Digital Signal Processing (DSP) circuits, programmable processors, Application Specific Integrated Circuits (ASICs), Field Programmable Gate Arrays (FPGAs), Complex Programmable Logic Devices (CPLDs), and so forth. These and other variations of the elements of system 200 are readily determined by one of ordinary skill in the art, given the teachings of the present invention provided herein, while maintaining the spirit of the present invention.

FIG. 4 shows an exemplary method 400 for phasing a polyploid using a daisy chain, in accordance with an embodiment of the present invention.

At step 405, provide a matrix 202 of sample organisms having rows that represent a respective one of the sample organisms and columns that represent, e.g., an ordered sequence of single-nucleotide polymorphisms (SNPs)).

At step 410, assemble haplotypes in polyploid genomes of two or more sample organisms (e.g., two or more sample organisms from matrix 202). Each of the haplotypes is a respective group of genes within any of the sample organisms that was inherited together from a single parent. At this point in method 400, the haplotypes are unphased haplotypes (that is, not correlated to a specific parent chromosome), and are assembled in preparation for phasing. In an embodiment, the term “haplotype” can refer to a set of single nucleo-tide polymorphisms (SNPs). In an embodiment, the term “haplotype” can refer to a collection of specific alleles (i.e., specific DNA sequences) in a cluster of tightly linked genes on a chromosome that are likely to be inherited together (i.e., likely to be conserved as a sequence that survives the descent of many generations of reproduction).

In an embodiment, step 410 can include step 410A.

At step 410A, assemble the haplotypes in a matrix of haplotypes 208.

At step 420, performing haplotype phasing on the haplotypes, to determine which parent chromosomes possess which of the haplotypes. The haplotype phasing can be performed in an iterative manner, where each iteration corresponds to a respective one of multiple phases of a haplotype phasing process, and wherein each of the multiple phases is performed to progressively result in obtaining a ploidy of 1.

It is to be appreciated that the present invention is not limited to any particular phasing process and, thus, can be applied to any phasing process as readily appreciated by one of ordinary skill in the art, given the teachings of the present invention provided herein. That is, irrespective of the phasing method used, various aspects of the present invention can be used to enhance the phasing results of any phasing process. The various aspects of the present invention that can be used for such purpose include, but are not limited to, one or more of the following: (i) reducing the ploidy p to floor(p/2) at each phase (step 420A); (ii) assign variables such that resulting intermediate ploids co-occur multiple times (420B); and (iii) use one or more daisy chain models to link ploids corresponding to the phasing process (420C).

In an embodiment, the phasing process is an algebraic phasing process, and the variables correspond to algebraic phasing process. The variables are used to, e.g., resolve the ploidy at various phases and/or for various equations and operations (e.g., intersection, relaxed intersection, union, complement, overlap) involved in the algebraic phasing. In such algebraic phasing, rules are applied to the input matrix D to find the minimum number of homozygous parents for the input matrix D. The goal of the phasing process is to determine the haplotype of the parent of the sample organisms in the input matrix.

In an embodiment, step 420 can include one or more of steps 420A-D.

At step 420A, in each of the multiple phases, reduce the ploidy p to floor(p/2). The ploidy p is the number of sets of chromosomes in a cell (of the sample organisms). The ploidy p can be reduced until the ploidy p is equal to one (that is, only mono-ploids remain) in all of the rows of matrix 202. This particular sub-step (420A) is based on the fact that samples in the input are related, and therefor at one point have share parents, hence the “/2”.

At step 420B, assign, in at least some of the multiple phases, variables in the haplotype phasing such that resulting intermediate ploids co-occur multiple times. The co-occurrence in the haplotypes define the links in the one or more daisy chain model(s) formed/linked per step 420C.

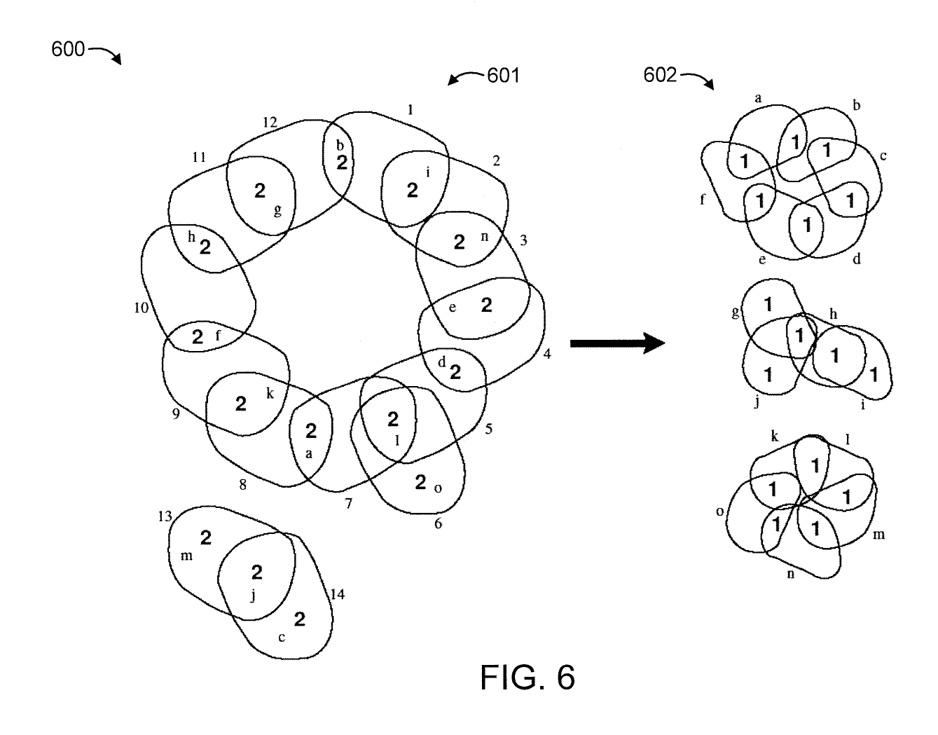

At step 420C, link, in each of the multiple phases, ploid samples together into one or more daisy chain models based on daisy chain linkage criteria. The daisy chain models can be used to obtain, e.g., in a final phase, the total number of haploids of the sample organism from a number of polyploids in a preceding phase(s). In the daisy chain model, ploid samples having a same ploidy are group together in one or more daisy chains. That is, the daisy chain linkage criteria for linking ploid samples together in a same daisy chain model includes the following: having the same ploidy. Ploidy reduction (e.g., 4-ploid to 2-ploid, 2-ploid to 1-ploid) is reflected in the ploid samples of each phase (where, per step 420A, each of the model samples for a given phase are reduced to floor(p/2)). Referring to FIG. 6, exemplary daisy chains 600 are shown, in accordance with an embodiment of the present invention. In particular, FIG. 6 shows a daisy chain model 601 on fourteen 4-ploid samples as an input to the haplotype phasing, and a daisy chain model 602 showing fifteen 2-ploid samples resulting from a first phase. In a next phase (not shown), the fifteen 2-ploids provide sixteen 1-ploids or haploids. Hence, this particular sub-step (420C) is used to reduce the space of the solution and attempts to minimize errors using the fact that the individuals share the link, that is, share haplotypes.

At step 420D, continue iterating to a next phase of the haplotype phasing process until one or more predetermined stop criterion is met. The criterion can include, but is not limited to, one or more of the following: (1) the input matrix only including monoploids; and (2) there is no change in the input matrix from one iteration/phase to the next iteration/phase.

At step 430, perform an action responsive to the total number of haploids of any (or both) of the one or more sample organisms or a metric (haplotype) derived therefrom.

In an embodiment, step 430 can include one or more of steps 430A-D.

At step 430A, perform a Genome wide association study. It an embodiment, the Genome wide association study can be used to determine if any gene variant is associated with a trait. In an embodiment, the Genome wide association study can involve identifying from which parent a haplotype has been inherited. Hence, in such a case, the present invention can provide a more fine-grained resolution output from a Genome wide association study than the prior art.

At step 430B, perform pedigree identification. For example, haplotypes phasing in the examination of different individuals can be used to determine possible relationships and the pedigree of the sample in the data.

At step 430C, impute missing values in a genetic marker for one of the sample organism based on information (e.g., results of the haplotype phasing) derived from or determined for the other sample organism. For example, using a correct phasing of a series of related individuals, it is possible to impute missing values in a genetic marker for a given one of the related individuals. This is often considered a fundamental task to properly analyze phenotype traits.

At step 430D, perform a medical test by a hardware device based on the results of the haplotype phasing. For example, based on the results of the haplotype phasing, a medical test can be performed for disease diagnosis, and so forth. That is, based on the results of the haplotype phasing indicating that a sample organism has a particular trait relating to a condition with which a particular disease is often associated, a medical test for that particular disease can be performed to determine its existence in the sample organism or a related sample organism.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK. C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

Having described preferred embodiments of a system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

1. A computer-implemented method for phasing polyploids, comprising:
assembling, by a processor, haplotypes in polyploid genomes of two or more sample organisms; and
performing, by the processor, haplotype phasing on the haplotypes using a haplotype phasing process having multiple phases applied to ploid samples corresponding to the haplotypes to determine which parent chromosomes possess which of the haplotypes;
wherein performing haplotype phasing includes
reducing, in each of the multiple phases, a ploidy p to a floor value (p/2);
assigning variables in at least some of the multiple phases such that resulting intermediate ones of the ploid samples co-occur multiple times in the at least some of the multiple phases; and
linking, in each of the multiple phases, the ploid samples together using one or more daisy chain models and daisy chain linkage criteria.

2. The computer-implemented method of claim 1, wherein, at each of the multiple phases of the haplotype phasing, said reducing step reduces the floor value to a result of dividing a current value of the ploidy p by two.

3. The computer-implemented method of claim 1, wherein at least one of the one or more daisy chain models comprises at least some of the co-occurring ones of the intermediate ploid samples, the at least some of the co-occurring ones of the intermediate ploid samples defining links in the one or more daisy chain models.

4. The computer-implemented method of claim 1, wherein the linkage criteria comprises linking together any of the ploid samples having a same ploidy value.

5. The computer-implemented method of claim 1, wherein a respective reduction resulting from said reduction step is represented in each of the one or more daisy chain models.

6. The computer-implemented method of claim 1, wherein a final one of the one or more daisy chain models consists of only haploids.

7. The computer-implemented method of claim 1, further comprising providing a matrix of sample organisms having rows that represent the two or more sample organisms and columns that represent various ones of the haplotypes.

8. The computer-implemented method of claim 7, wherein the haplotypes comprise single-nucleotide polymorphisms (SNPs).

9. The computer-implemented method of claim 7, wherein the haplotypes comprise alleles.

10. The computer-implemented method of claim 1, wherein said assembling step assembles the haplotypes in a matrix of haplotypes.

11. The computer-implemented method of claim 1, wherein the haplotype phasing comprises applying algebraic rules to the haplotypes.

12. The computer-implemented method of claim 11, wherein the algebraic rules are applied with respect to a data structure storing the haplotypes.

13. The computer-implemented method of claim 1, further comprising imputing missing values in a genetic marker for one of the two or more sample organisms based on a result of the haplotype phasing.

14. A computer program product for phasing polyploids, the computer program product comprising a non-transitory computer readable storage medium having program instructions embodied therewith, the program instructions executable by a computer to cause the computer to perform a method comprising:
assembling, by a processor, haplotypes in polyploid genomes of two or more sample organisms; and
performing, by the processor, haplotype phasing on the haplotypes using a haplotype phasing process having multiple phases applied to ploid samples corresponding to the haplotypes to determine which parent chromosomes possess which of the haplotypes;
wherein performing haplotype phasing includes
reducing, in each of the multiple phases, a ploidy p to a floor value (p/2);
assigning variables in at least some of the multiple phases such that resulting intermediate ones of the ploid samples co-occur multiple times in the at least some of the multiple phases; and
linking, in each of the multiple phases, the ploid samples together using one or more daisy chain models and daisy chain linkage criteria.

15. The computer-implemented method of claim 14, wherein, at each of the multiple phases of the haplotype phasing, said reducing step reduces the floor value to a result of dividing a current value of the ploidy p by two.

16. The computer-implemented method of claim 14, wherein at least one of the one or more daisy chain models comprises at least some of the co-occurring ones of the intermediate ploid samples, the at least some of the co-occurring ones of the intermediate ploid samples defining links in the one or more daisy chain models.

17. The computer-implemented method of claim 14, wherein the linkage criteria comprises linking together any of the ploid samples having a same ploidy value.

18. The computer-implemented method of claim 14, wherein a respective reduction resulting from said reduction step is represented in each of the one or more daisy chain models.

19. The computer-implemented method of claim 14, wherein a final one of the one or more daisy chain models consists of only haploids.

20. A computer processing system for phasing polyploids, comprising:
a processor, configured to
assemble haplotypes in polyploid genomes of two or more sample organisms; and
perform haplotype phasing on the haplotypes using a haplotype phasing process having multiple phases applied to ploid samples corresponding to the haplotypes to determine which parent chromosomes possess which of the haplotypes;
wherein the processor performs the haplotype phasing by
reducing, in each of the multiple phases, a ploidy p to a floor value (p/2);
assigning variables in at least some of the multiple phases such that resulting intermediate ones of the ploid samples co-occur multiple times in the at least some of the multiple phases; and
linking, in each of the multiple phases, the ploid samples together using one or more daisy chain models and daisy chain linkage criteria.
